(19) United States
(12) Patent Application Publication
(10) Pub. No.: US 2026/0092866 A1
(43) Pub. Date: Apr. 2, 2026

(54) FLUORESCENCE-BASEDTOXIN DETECTION APPARATUS AND METHOD

(71) Applicant: United States of America as Represented by The Secretary of The Army, Alexandria, VA (US)

(72) Inventors: Luke A. Gurtowski, Madison, MS (US); Angela M. Evans, Jackson, MS (US); Charles H. Laber, Edwards, MS (US); Brett A. Hayhurst, Trumansburg, NY (US); Karen G. Keil, Wilson, NY (US); Chris S. Griggs, Vicksburg, MA (US)

(73) Assignee: United States of America as Represented by The Secretary of The Army, Alexandria, VA (US)

(21) Appl. No.: 18/901,426

(22) Filed: Sep. 30, 2024

(51) Int. Cl. G01N 21/64; G01N 33/543; G01N 33/68

(52) U.S. Cl. CPC G01N 21/6428; G01N 33/54313; G01N 33/582; G01N 2021/6439; G01N 2021/6443; G01N 2021/6471; G01N 2333/405

(57) ABSTRACT

In one embodiment, a fluorescence-based kit for detection and analysis of a toxin in a liquid sample includes a diluted antibody mixture in a preparation container to receive the liquid sample to produce a preparation solution; a fluorescent compound absorbed onto colloidal graphene in an analysis container to receive the preparation solution to produce an analysis solution; and a portable detector including one or more reference solutions each having a different one of a plurality of known levels of toxin concentration of the toxin, and a light source to shine a light onto the analysis solution and the one or more reference solutions.

### STATEMENT OF GOVERNMENT INTEREST

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees.

### BACKGROUND

### Field of the Invention

The present invention relates to fluorescence-based systems and methods of detecting and analyzing toxins and, more specifically, rapid detection and analysis of toxins such as harmful algal bloom (HAB) toxins.

### Description of the Related Art

This section introduces aspects that may help facilitate a better understanding of the invention. Accordingly, the statements of this section are to be read in this light and are not to be understood as admissions about what is prior art or what is not prior art.

Harmful algal blooms (HABs) occur when algae grow out of control and sometimes produce toxins harmful to people and animals. HABs present a significant threat to fresh waters, necessitating routine testing to protect humans from exposure to contaminated drinking and recreational waters and for forecasting and modelling. Because toxin profiles change spatially and temporally, there is a significant need for rapid tests that can provide real-time, local answers. The dynamic nature of HABs can result in an unexpected emergence of dangerous toxin levels in areas that are otherwise considered “low risk” for toxin intrusion. Consequently, they may receive little or no regular testing to the detriment of the communities in those areas.

### SUMMARY

A technological gap currently exists in that toxins related to harmful algal blooms (HABs) cannot be rapidly quantified in the field. Instead, samples must be shipped and then tested in laboratories. Thus, delays occur regarding analysis and reporting.

The present invention was developed to close this technological gap by providing a field-portable graphene-based technology for rapid and economical visual HAB-toxin detection via fluorescence analysis using a fluorescent probe containing a fluorescent compound.

Embodiments of the invention are directed to systems and method for rapid detection and semi-quantitative visual analysis of microcystin-LR (MC-LR), a toxin related to HABs, via fluorescence of dispersions with graphene and a tagged probe specific to the process.

An aspect of the invention is directed to a fluorescence-based kit for detection and analysis of a toxin in a liquid sample. The fluorescence-based kit comprises a diluted antibody mixture in a preparation container to receive the liquid sample to produce a preparation solution; a fluorescent compound absorbed onto colloidal graphene in an analysis container to receive the preparation solution to produce an analysis solution; and a portable detector including one or more reference solutions each having a different one of a plurality of known levels of toxin concentration of the toxin, and a light source to shine a light onto the analysis solution and the one or more reference solutions.

Another aspect is directed to a fluorescence-based method for detection and analysis of a toxin in a liquid sample. The method comprises: combining a diluted antibody mixture and the liquid sample to produce a preparation solution; combining a fluorescent compound absorbed onto colloidal graphene and the preparation solution to produce an analysis solution; shining a light onto the analysis solution and one or more reference solutions each having a different one of a plurality of known levels of toxin concentration of the toxin; observing a fluorescence intensity/signal of the analysis solution from exposure to the light to detect whether the toxin is present in the analysis solution; and comparing the fluorescence intensity/signal of the analysis solution from exposure to the light with one or more fluorescence intensities/signals of the one or more reference solutions from exposure to the light to analyze a level of the toxin that is present in the analysis solution.

### BRIEF DESCRIPTION OF THE DRAWINGS

Embodiments of the invention will become more fully apparent from the following detailed description, the appended claims, and the accompanying drawings in which like reference numerals identify similar or identical elements.

FIG. 1 is a schematic illustration of preparing a potentially toxin-contaminated water as an analysis solution for fluorescence-based detection and analysis.

FIG. 2 is a schematic illustration of detecting and analyzing a number of analysis solutions.

FIG. 3 is a graphical plot illustrating fluorescence intensities at different light wavelengths for analysis solutions at a plurality of different toxin concentrations.

FIG. 4 shows an example of a qualitative visual comparison of fluorescence as an indication of toxin concentrations from 0 ppb (blank) to 1000 ppb in natural light.

FIG. 5 shows examples of a qualitative visual comparison of fluorescence as an indication of toxin concentrations, (A) without a filter in natural light, (B) without a filter under a blue light at 470 nm wavelength, and (C) with an orange filter under a blue light at 470 nm wavelength.

FIG. 6A is a perspective view of an example of a housing for a fluorescence-based toxin detection system or apparatus.

FIG. 6B is a top plan view of the housing.

FIG. 6C is a front elevational view of the housing.

FIG. 7A is a perspective view of an example of a fluorescence-based toxin detection system or apparatus before loading the reference and analysis solutions.

FIG. 7B is a perspective view of the fluorescence-based toxin detection apparatus after loading the reference and analysis solutions.

FIG. 8 is a flow diagram illustrating an example of a fluorescence-based method for detection and analysis of a toxin in a liquid sample.

### DETAILED DESCRIPTION

Detailed illustrative embodiments of the present invention are disclosed herein.

However, specific structural and functional details disclosed herein are merely representative for purposes of describing example embodiments of the present invention. The present invention may be embodied in many alternate forms and should not be construed as limited to only the embodiments set forth herein. Further, the terminology used herein is for the purpose of describing particular embodiments only and is not intended to be limiting of example embodiments of the invention.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

One feature of this invention is to provide a field-portable graphene-based technology for rapid visual HAB-toxin detection via fluorescence analysis using a fluorescent probe. This technology provides semi-quantitative visual analysis of microcystin-LR (MC-LR), a toxin related to HABs, via fluorescence of dispersions with graphene and a tagged probe specific to the process.

In embodiments, the process and technology of interest provide visual detection and semi-quantification of HAB-toxin MC-LR in freshwater samples. The technology involves a probe consisting of a fluorescent compound that is adsorbed onto colloidal graphene. Upon addition of a freshwater sample and intermediate processing, the fluorescent compound is released from the graphene into the solution containing the sample to produce an analysis solution. The amount of this fluorescent compound that is released is correlated to the concentration of MC-LR in the freshwater sample. Because this compound fluoresces when exposed to certain wavelengths of light, the final solution may provide a visual signal based upon the presence and concentration of MC-LR in the freshwater sample.

Colloidal graphene has been used, for instance, as a transducer in a homogeneous fluorescence-based immunosensor for rapid and sensitive analysis of MC-LR. To facilitate the ligation between DNA oligomers and MC-LR, no self-dimer or hairpin structure is formed between or in the designed DNA oligomers. The preparation involves the use of monoclonal anti-MC-LR antibody (Clone MC8C10), MC-LR (Minimum 95% by HPLC), human IgG, bovine serum albumin (BSA), monoclonal anti-human AFP (anti-AFP), goat anti-human IgM (anti-IgM), mouse anti-human IgG (anti-IgG), N-Succinimidyl 3-(2-pyridyldithio)-propionate (SPDP), other analytical grade reagents, and ultrapure water.

Graphene Oxide (GO) can be prepared according to a modified Hummers method. For instance, 1.0 g graphite powder is first dispersed in 23 mL H₂SO₄ and stirred for 12 hours at 25° C. Then 3 g KMnO₄ can be slowly added to the above mixture under vigorous stirring at 0° C. After being sonicated for 12 hours, 46 mL ultrapure water is added to the mixture and heated to boil for 20 minutes. Then, the reaction is terminated by the addition of the mixtures of 140 mL ultrapure water and 10 mL H₂O₂ (30%). Finally, the as-prepared GO is separated and purified by centrifugation, and washed with 5% HCl and ultrapure water for several times. An environment-friendly hydrothermal route can be used to convert GO into colloidal graphene. In a typical experiment, 50 mL of 100 mg/L GO is transferred to a Teflon-lined autoclave and heated at 180° C. for 8 hours. After being cooled to room temperature, the obtained colloidal graphene is stored at 4° C.

For another example of colloidal graphene, see Meng Liu et al., “Colloidal Graphene as a Transducer in Homogeneous Fluorescence-Based Immunosensor for Rapid and Sensitive Analysis of Microcystin-LR,” Environmental Science & Technology, 2012, 46, 12567-12574, which is incorporated herein by reference in its entirety.

### Fluorescence-based Technique

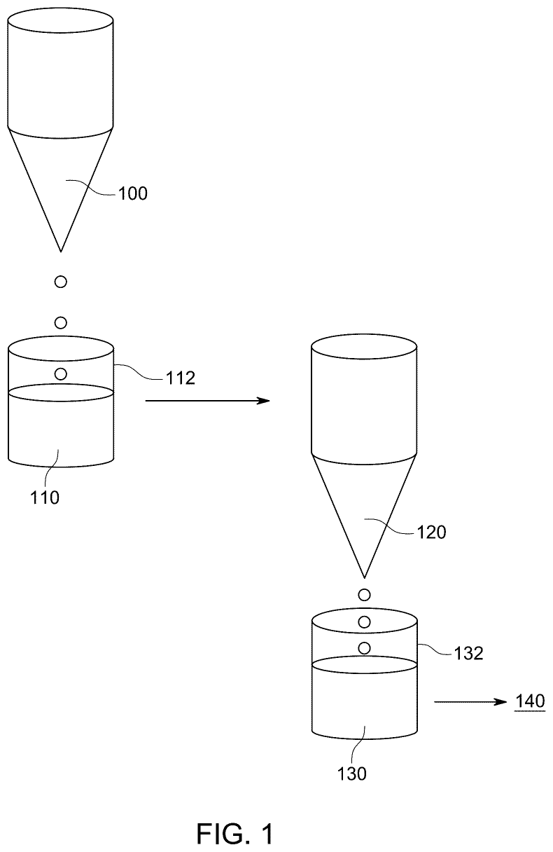

FIG. 1 is a schematic illustration of preparing a potentially toxin-contaminated water as an analysis solution for fluorescence-based detection and analysis. A sample of the potentially toxin-contaminated water 100 is introduced into a diluted antibody mixture 110 in a preparation vial or preparation container 112 to produce a preparation solution 120, typically within a few minutes. The diluted antibody mixture 110 may include an antibody of microcystin-LR (MC-LR) diluted in DI water. The preparation solution 120 is then introduced into a fluorescent compound absorbed onto colloidal graphene 130 in an analysis vial or analysis container 132 to produce an analysis solution 140. The analysis container 132 may be made of a clear glass or clear polymer to facilitate fluorescence-based visual analysis of the analysis solution 140 contained therein.

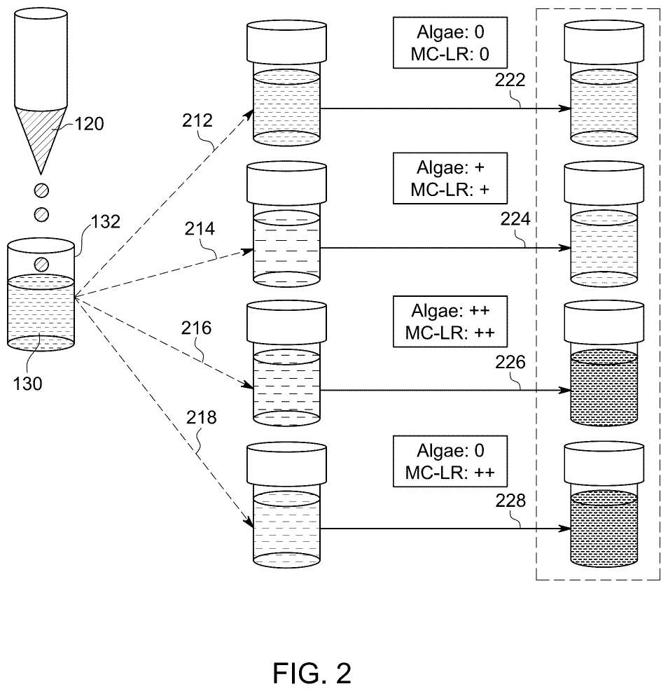

FIG. 2 is a schematic illustration of detecting and analyzing a number of analysis solutions. The first analysis solution 212 has no algae and no HAB-toxin MC-LR. The second analysis solution 214 has algae and MC-LR. The third analysis solution 216 has a greater concentration of algae and MC-LR than the second analysis solution. The fourth analysis solution 218 has no algae and the same concentration of MC-LR as the third analysis solution 216.

The analysis solutions are exposed to a light having a wavelength to produce fluorescence intensities/signals from toxins in the analysis solutions. The first analysis solution 212 has a zero first fluorescence intensity/signal 222 because the toxin concentration is zero.

The second analysis solution 214 has a second fluorescence intensity/signal 224 with an intensity corresponding to the concentration of the toxin in the second analysis solution. The third analysis solution 216 has a third fluorescence intensity/signal 226 with a higher intensity than that of the second analysis solution 214 corresponding to the higher concentration of the toxin in the third analysis solution. The fourth analysis solution 218 has a fourth fluorescence intensity/signal 228 with the same intensity as that of the third analysis solution 216 because they have the same concentration of MC-LR even though the fourth analysis solution 218 contains no algae.

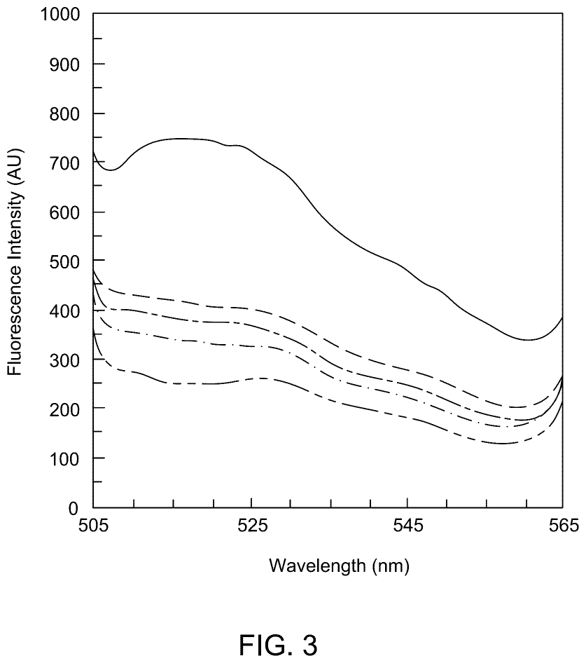

FIG. 3 is a graphical plot illustrating fluorescence intensities at different light wavelengths for analysis solutions at a plurality of different toxin concentrations. While FIG. 2 demonstrates a qualitative, visual comparison of fluorescence as an indication of toxin concentration, FIG. 3 shows a quantitative analysis of toxin concentration based on fluorescence intensity data as a function of light wavelengths.

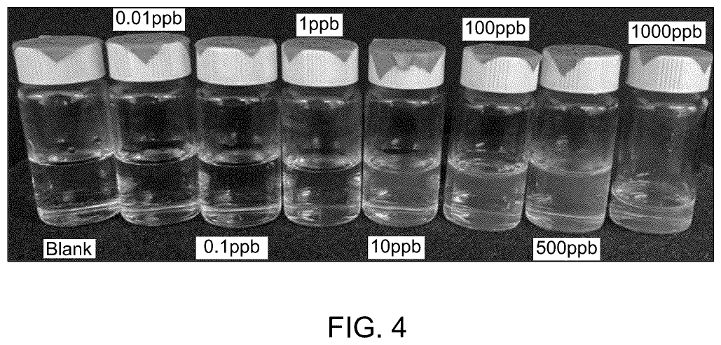

FIG. 4 shows an example of a qualitative visual comparison of fluorescence as an indication of toxin concentrations from 0 ppb (blank) to 1000 ppb in natural light. This provides the basis for a visual (qualitative or semi-qualitative) analysis of toxin concentration in a sample.

The research team has exposed toxin samples to light with different wavelengths and viewed with different colored filters (red, orange, and yellow). The images of the exposed toxin samples are observed for visual analysis. The results show that blue lights between 450 nm and 495 nm, more specifically 470 nm, provide the greatest illumination, particularly when viewed with an orange-colored filter. A light at a wavelength below 450 nm also can cause the samples to illuminate, but a light at a wavelength of 520 nm or greater does not have a noticeable effect. In short, a light at a wavelength of 365 nm to 495 nm may be used to enhance the visual analysis, while a light at a wavelength of 450 nm to 495 nm, more specifically 470 nm, can produce the greatest illumination or enhancement. The lights and filters for this analysis are available, for instance, from FoxFury, as a kit such as HammerHead H7A FS Forensic Light Kit.

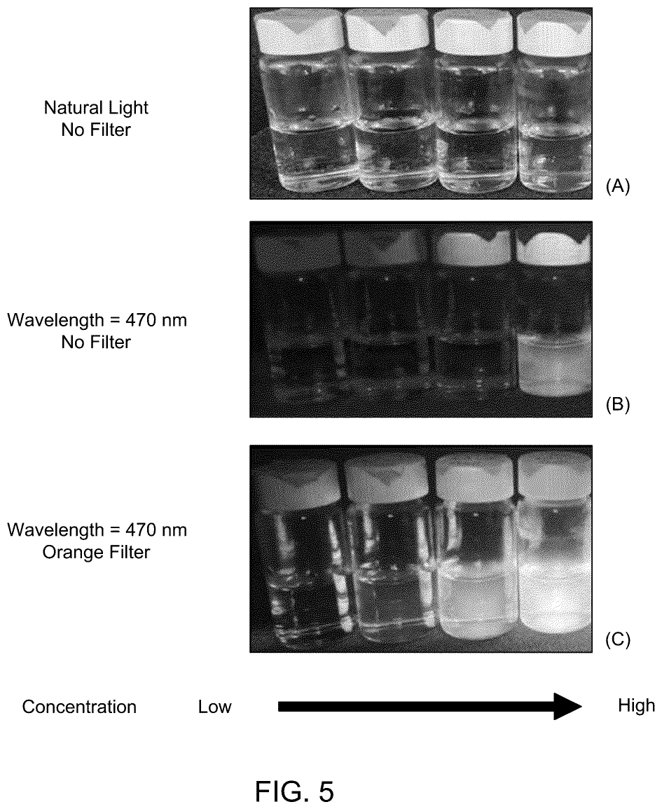

FIG. 5 shows examples of a qualitative visual comparison of fluorescence as an indication of toxin concentrations, (A) without a filter in natural light, (B) without a filter under a blue light at 470 nm wavelength, and (C) with an orange filter under a blue light at 470 nm wavelength. The use of light and filter can enhance the visual analysis in the fluorescence-based technique. The colored filter may be provided in the form of color tinted glasses or eyewear to be worn by the observer. Experiments have shown that the use of blue light between 450 nm and 495 nm, and more specifically 470 nm, and/or the use of an orange filter, produces the unexpected result of far superior visual enhancement for HAB toxins, especially when an orange filter is used in conjunction with a 470 nm light. The visual analysis significantly reduces the barrier for use based upon skill, as minimal training will be necessary to utilize the technology. Additionally, more rapid detection of toxins will be permitted relative to currently available analytical techniques.

### Fluorescence-based System

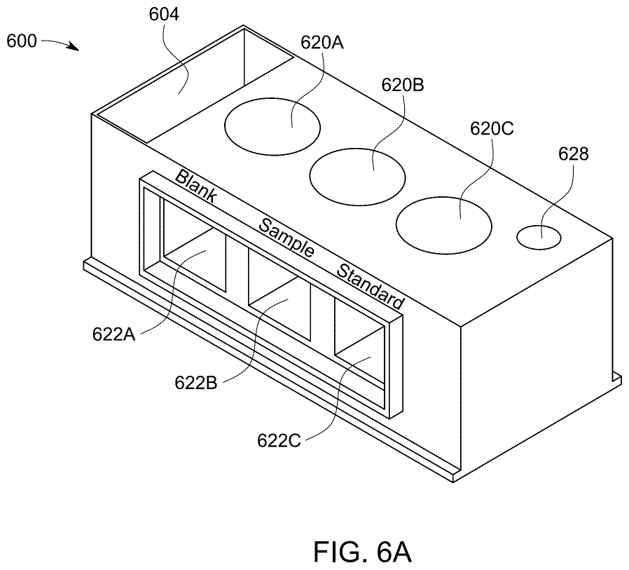

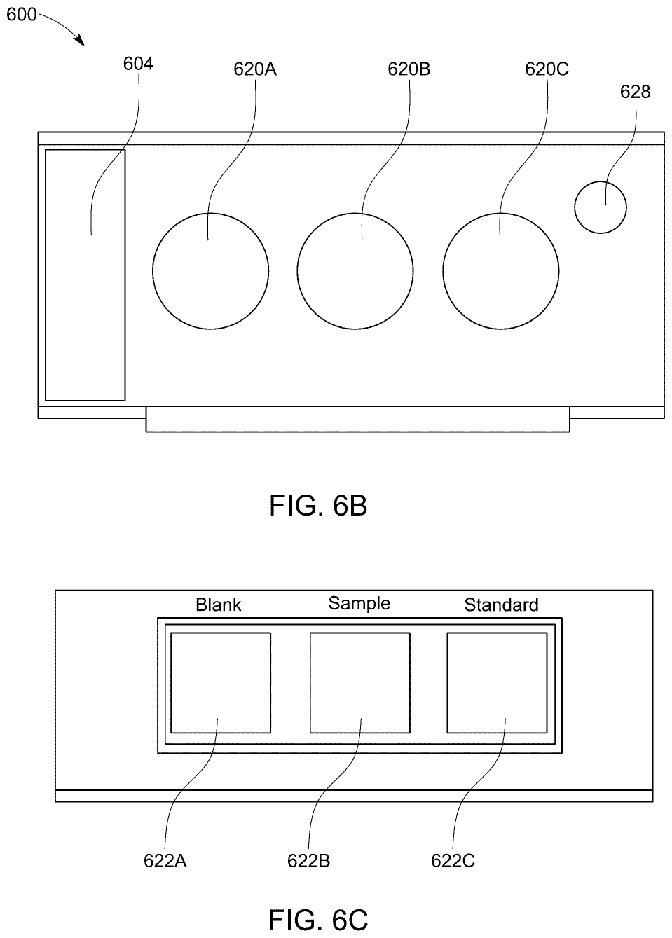

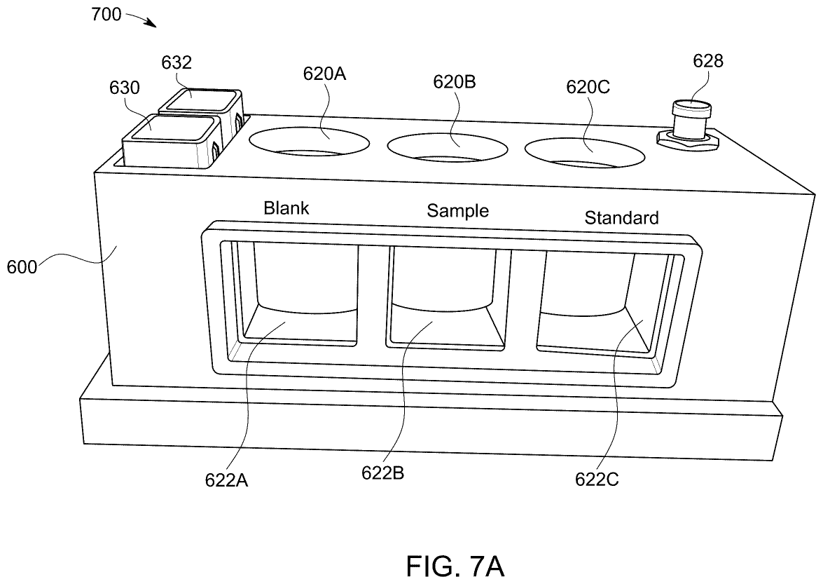

FIG. 6A is a perspective view of an example of a housing 600 for a fluorescence-based toxin detection system or apparatus. FIG. 6B is a top plan view of the housing. FIG. 6C is a front elevational view of the housing. The housing 600 includes a plurality of container slots 620 and corresponding viewing openings/windows 622. A cavity or chamber 604 is provided for power supply. In this example, the housing 600 includes three container slots 620A, 620B, 620C with three corresponding windows 622A, 622B, 622C. The housing 600 is a hand portable detector housing in specific embodiments.

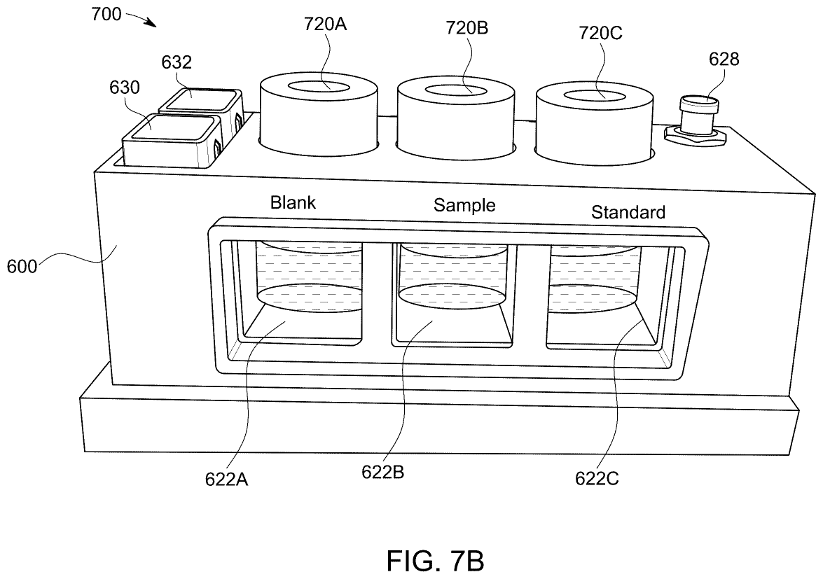

FIG. 7A is a perspective view of an example of a fluorescence-based toxin detection system or apparatus 700 before loading the reference and analysis solutions. FIG. 7B is a perspective view of the fluorescence-based toxin detection apparatus 700 after loading the reference and analysis solutions. A light control/button 628 is provided to control the use of light from a light source to illuminate reference fluids/solutions and analysis fluids/solutions. Batteries 630, 632 are provided in the power supply cavity or chamber 604 to power the apparatus 700.

The three container slots 620A, 620B, 620C correspond to three windows 622A, 622B, 622C. The first slot 620A is configured to receive a first reference container 720A, the second slot 620B is configured to receive an analysis container 720B (similar to or same as the analysis container 132 in FIGS. 1 and 2), and the third slot 620C is configured to receive a second reference container 720C. In one example, the first reference container 720A contains a blank (zero toxin concentration) fluid and the second reference container 720C contains a standard fluid having a known level of toxin concentration. In another example, the first reference container 720A contains a first reference or comparison solution having a first level of toxin concentration and the second reference container 720C contains a second reference or comparison solution having a second level of toxin concentration which is a different level of toxin concentration from the first level (e.g., higher level of toxin concentration). The reference/comparison containers 720A, 720C may be made of a clear glass or clear polymer to facilitate fluorescence-based visual observation of the reference/comparison solutions contained therein.

The first reference container 720A and the second reference container 720C are easily and readily replaceable. One approach is to select a first reference container 720A containing a first reference solution having a first level of toxin concentration which is darker or less illuminated than the analysis solution and a second reference container 720C containing a second reference solution having a second level of toxin concentration which is brighter or more illuminated than the analysis solution. The level of toxin concentration of the analysis solution is between the first level and the second level, thereby providing a rapid semi-quantitative visual analysis of an analysis solution.

The toxin detection apparatus 700 can be operated as a visual fluorometer for toxin concentration. An embodiment is directed to a visual fluorometer kit or a fluorescence-based visual toxin detection kit which may include, without limitation, the housing 600, the analysis container or analysis vial 720B, a plurality of reference containers (e.g., 720A, 720C) for containing a plurality of reference solutions at a variety of toxin concentration levels, at least one colored filter or at least one pair of color tinted glasses, etc. The analyzed solution will be analyzed within a colorless, transparent (glass) 20-mL scintillation vial that will be illuminated with LEDs at 470 nm located below the viewing windows of the apparatus (600). The system is powered with 9V batteries (630 and 632) and operated by pressing down the button 628.

### Fluorescence-based Method

Embodiments of the invention are directed to a standardized process of providing the fluorescence intensity/signal for visual analysis. Graphene-based dispersions containing the fluorescent probe are systematically exposed to freshwater samples within a vial or similar container. Then, these samples are placed in a specialized device such as the fluorescence-based toxin detection apparatus 700 and exposed to light of a relevant wavelength (e.g., 470 nm) for visual analysis to determine if the samples glow or provide other observable signals.

Visual aids, including colored (e.g., orange and red) filters for the device or color tinted eyewear, may be used for efficient review or enhanced analysis of the sample.

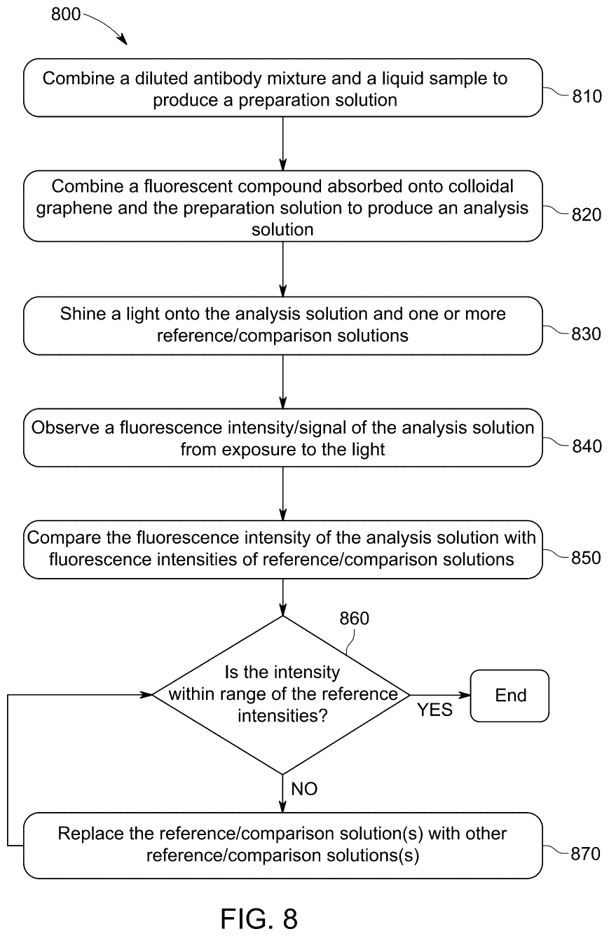

FIG. 8 is a flow diagram illustrating an example of a fluorescence-based method 800 for detection and analysis of a toxin in a liquid sample. Step 810 involves combining a diluted antibody mixture and the liquid sample to produce a preparation solution, e.g., in a preparation container or vial. This typically takes a few minutes. In step 820, a fluorescent compound absorbed onto colloidal graphene and the preparation solution are combined to produce an analysis solution, e.g., in an analysis container or vial, which may be made of a clear glass or clear polymer to facilitate fluorescence-based visual analysis of the analysis solution contained therein.

To prepare an analysis solution, a toxin-contaminated water can be added to the sample vial or container 720B containing a graphene-assisted probe that fluoresces based upon toxin concentration. The graphene-assisted probe used for fluorescence analysis may employ any suitable fluorescent compound. An example is the fluorescently tagged oligomer (/5ThioMC6-D/TT GGT TCT GT/36-FAM/) that is used to synthesize the probe for fluorescence analysis. The probe may be prepared using HPLC (High Performance Liquid Chromatography) purification or standard desalting purification. Both have been shown to perform satisfactorily in the present technique.

Step 830 involves shining a light onto the analysis solution and one or more reference solutions each having a different one of a plurality of known levels of toxin concentration of the toxin. This may utilize the use of the housing 600 for holding the first reference/comparison container 720A containing the first reference/comparison solution, the analysis container 720B containing the analysis solution, and the second reference/comparison container 720C containing the second reference/comparison solution. For best results, the ambient light is kept as low as possible. The wavelength of the light may have a default setting that can be changed readily. The available wavelength may be between 365 nm and 495 nm, or may be set for a blue light of between 450 nm and 495 nm. The default setting may be 470 nm.

Step 840 involves observing or recording a fluorescence intensity/signal of the analysis solution from exposure to the light. This can be used to detect whether the toxin is present in the analysis solution. The observation may utilize the three windows 622A, 622B, 622C of the housing 600.

An optional step may be included for enhancing the fluorescence intensity/signal of the analysis solution and the one or more fluorescence intensities/signals of the one or more reference solutions using a colored filter through which to view the analysis solution and the one or more reference solutions exposed to the light. The colored filter may be selected from one or more colored filters including an orange filter, a red filter, or a yellow filter. The apparatus may be equipped with an orange filter as a default setting and can be configured to change/replace the colored filter readily. Another optional step may involve changing the wavelength of the light and/or changing the colored filter to enhance or optimize the fluorescence illumination.

Step 850 involves comparing the fluorescence intensity/signal of the analysis solution from exposure to the light with one or more fluorescence intensities/signals of the one or more reference solutions from exposure to the light to analyze a level of the toxin that is present in the analysis solution. In one example, the first reference solution is a blank solution at zero toxin concentration and the second reference solution has a standard, known level of toxin concentration. In another example, the first reference solution has a first level of toxin concentration, and the second reference solution has a second level of toxin concentration which is higher than the first level. If the fluorescence intensity of the analysis solution matches the intensity of any of the reference solutions, the level of toxin concentration with the matched intensity can be recorded as the estimated level of toxin concentration for the analysis solution. If the fluorescence intensity of the analysis solution is between the intensities of the first and second reference solutions, the estimated level of toxin concentration for the analysis solution can be recorded as falling within a range between the first level and the second level of toxin concentration.

In step 860, the method determines whether the fluorescence intensity of the analysis solution either matches the intensity of any of the reference solutions or falls within a range between the levels of intensity of the first and second reference solutions. If yes, the process ends.

If the fluorescence intensity of the analysis solution does not match the intensity of any of the reference solutions and is not between the levels of intensity of the first and second reference solutions, one or more of the reference solutions are replaced by other reference solutions at different known levels of toxin concentration in step 870. This is repeated until the condition of step 860 is met, and the process ends with an estimate of the toxin concentration of the analysis solution.

In addition, the method may include the optional step of adjusting the wavelength of the light and changing the colored filter to increase enhancement or optimize enhancement of the fluorescence intensity. This will improve the visual analysis of the method.

Furthermore, step 850 may be modified to include comparing the fluorescence intensity/signal of the analysis solution from exposure to the light with a plurality of fluorescence intensities/signals of a plurality of reference solutions from exposure to the light; identifying a first fluorescence intensity of a first reference solution which is less illuminated than the analysis solution and a second fluorescence intensity of a second reference solution which is more illuminated than the analysis solution; and estimating that the analysis solution has a toxin concentration between a first toxin concentration of the first reference solution and a second toxin concentration of the second reference solution. In one example, the first reference solution has zero toxin concentration, and the second reference solution has a positive toxin concentration. In another example, the first reference solution is identified to be less illuminated than the analysis solution but not less illuminated than any other reference solution of the plurality of reference solutions which are less illuminated than the analysis solution; and the second reference solution is identified to be more illuminated than the analysis solution but not more illuminated than any other reference solution of the plurality of reference solutions which are more illuminated than the analysis solution. This helps narrow the range of the toxin concentration for the analysis solution between the first toxin concentration and the second toxin concentration as much as possible under the circumstances.

This technology can be adapted for use by government partners (e.g. USACE, EPA, and NOAA). Additionally, this technology can be licensed to private-sector firms that specialize in environmental or chemical analytical technologies (e.g., Hach, ThermoFisher Scientific, etc.). The overall use of this technology would be for freshwater analysis of HAB toxins.

The detection system may be provided as a fluorescence-based kit for detection and analysis of a toxin in a liquid sample. The kit may include a diluted antibody mixture in a preparation container to receive the liquid sample to produce a preparation solution; a fluorescent compound absorbed onto colloidal graphene in an analysis container to receive the preparation solution to produce an analysis solution; a portable detector including one or more reference solutions each having a different one of a plurality of known levels of toxin concentration of the toxin, and a light source to shine a light onto the analysis solution and the one or more reference solutions; and one or more colored filters through which to view the analysis solution and the one or more reference solutions exposed to the light.

Embodiments of the invention can be manifest in the form of methods, kits, and apparatuses for practicing those methods. As compared to prior approaches, this detection and analysis apparatus and method have a number of distinguishing features. For instance, the detection produces rapid results in a cost-effective manner. In addition, the detection is not merely qualitative. The visual fluorescence-based analysis is semi-quantitative by comparing fluorescence intensity of the sample analysis solution with the known fluorescence intensities of a plurality of reference or comparison solutions to obtain an estimated toxin concentration.

As will be appreciated by one of ordinary skill in the art, the present invention may be embodied as an apparatus (including, for example, a system, a machine, a device, and/or the like), as a method (including, for example, a business process, and/or the like), as a computer-readable storage medium, or as any combination of the foregoing.

The inventive concepts taught by way of the examples discussed above are amenable to modification, rearrangement, and embodiment in several ways. Accordingly, although the present disclosure has been described with reference to specific embodiments and examples, persons skilled in the art will recognize that changes may be made in form and detail without departing from the spirit and scope of the disclosure.

An interpretation under 35 U.S.C. § 112(f) is desired only where this description and/or the claims use specific terminology historically recognized to invoke the benefit of interpretation, such as “means,” and the structure corresponding to a recited function, to include the equivalents thereof, as permitted to the fullest extent of the law and this written description, may include the disclosure, the accompanying claims, and the drawings, as they would be understood by one of skill in the art.

To the extent the subject matter has been described in language specific to structural features and/or methodological steps, it is to be understood that the subject matter defined in the appended claims is not necessarily limited to the specific features or steps described.

Rather, the specific features and steps are disclosed as example forms of implementing the claimed subject matter. To the extent headings are used, they are provided for the convenience of the reader and are not to be taken as limiting or restricting the systems, techniques, approaches, methods, devices to those appearing in any section. Rather, the teachings and disclosures herein can be combined, rearranged, with other portions of this disclosure and the knowledge of one of ordinary skill in the art. It is the intention of this disclosure to encompass and include such variation.

The indication of any elements or steps as “optional” does not indicate that all other or any other elements or steps are mandatory. The claims define the invention and form part of the specification. Limitations from the written description are not to be read into the claims.

Unless explicitly stated otherwise, each numerical value and range should be interpreted as being approximate as if the word “about” or “approximately” preceded the value or range.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

What is claimed is:

1. A fluorescence-based kit for detection and analysis of a toxin in a liquid sample, the fluorescence-based kit comprising:
a diluted antibody mixture in a preparation container to receive the liquid sample to produce a preparation solution;
a fluorescent compound absorbed onto colloidal graphene in an analysis container to receive the preparation solution to produce an analysis solution; and
a portable detector including one or more reference solutions each having a different one of a plurality of known levels of toxin concentration of the toxin, and a light source to shine a light onto the analysis solution and the one or more reference solutions.

2. The fluorescence-based kit of claim 1, wherein the portable detector further includes:
one or more colored filters through which to view the analysis solution and the one or more reference solutions exposed to the light.

3. The fluorescence-based kit of claim 2,
wherein one or more colored filters include one or more of an orange filter, a red filter, or a yellow filter.

4. The fluorescence-based kit of claim 1,
wherein the light of the light source has a wavelength of 365 to 495 nm.

5. The fluorescence-based kit of claim 1,
wherein the light of the light source has a wavelength of 450 to 495 nm.

6. The fluorescence-based kit of claim 1,
wherein the light of the light source has a wavelength of 470 nm.

7. The fluorescence-based kit of claim 6, wherein the portable detector further includes:
an orange filter through which to view the analysis solution and the one or more reference solutions exposed to the light at 470 nm wavelength.

8. The fluorescence-based kit of claim 1,
wherein the diluted antibody mixture comprises an antibody of microcystin-LR (MC-LR) diluted in DI water.

9. A fluorescence-based method for detection and analysis of a toxin in a liquid sample, the method comprising:
combining a diluted antibody mixture and the liquid sample to produce a preparation solution;
combining a fluorescent compound absorbed onto colloidal graphene and the preparation solution to produce an analysis solution;
shining a light onto the analysis solution and one or more reference solutions each having a different one of a plurality of known levels of toxin concentration of the toxin;
observing a fluorescence intensity of the analysis solution from exposure to the light to detect whether the toxin is present in the analysis solution; and
comparing the fluorescence intensity of the analysis solution from exposure to the light with one or more fluorescence intensities of the one or more reference solutions from exposure to the light to analyze a level of the toxin that is present in the analysis solution.

10. The method of claim 9, further comprising:
enhancing the fluorescence intensity of the analysis solution and the one or more fluorescence intensities of the one or more reference solutions using a colored filter through which to view the analysis solution and the one or more reference solutions exposed to the light.

11. The method of claim 10, further comprising:
selecting the colored filter from one or more colored filters including an orange filter, a red filter, or a yellow filter.

12. The method of claim 9, further comprising:
selecting a wavelength of the light between 365 nm and 495 nm.

13. The method of claim 9, further comprising:
selecting a wavelength of the light between 450 nm and 495 nm.

14. The method of claim 9, further comprising:
selecting a wavelength of the light of 470 nm.

15. The method of claim 14, further comprising:
enhancing the fluorescence intensity of the analysis solution and the one or more fluorescence intensities of the one or more reference solutions using an orange-colored filter through which to view the analysis solution and the one or more reference solutions exposed to the light at 470 nm wavelength.

16. The method of claim 9, further comprising:
enhancing the fluorescence intensity of the analysis solution and the one or more fluorescence intensities of the one or more reference solutions using a colored filter through which to view the analysis solution and the one or more reference solutions exposed to the light; and
adjusting a wavelength of the light and changing the colored filter to increase enhancement of the fluorescence intensity.

17. The method of claim 9,
wherein the diluted antibody mixture comprises an antibody of microcystin-LR (MC-LR) diluted in DI water which is combined with the liquid sample to produce the preparation solution.

18. The method of claim 9, wherein comparing the fluorescence intensity of the analysis solution from exposure to the light comprises:
comparing the fluorescence intensity of the analysis solution from exposure to the light with a plurality of fluorescence intensities of a plurality of reference solutions from exposure to the light;
identifying a first fluorescence intensity of a first reference solution which is less illuminated than the analysis solution and a second fluorescence intensity of a second reference solution which is more illuminated than the analysis solution; and
estimating that the analysis solution has a toxin concentration between a first toxin concentration of the first reference solution and a second toxin concentration of the second reference solution.

19. The method of claim 18,
wherein the first reference solution has zero toxin concentration and the second reference solution has a positive toxin concentration.

20. The method of claim 18,
wherein the first reference solution is identified to be less illuminated than the analysis solution but not less illuminated than any other reference solution of the plurality of reference solutions which are less illuminated than the analysis solution; and
wherein the second reference solution is identified to be more illuminated than the analysis solution but not more illuminated than any other reference solution of the plurality of reference solutions which are more illuminated than the analysis solution.
